(19) United States
(12) Patent Application Publication
(10) Pub. No.: US 2007/0106230 A1
(43) Pub. Date: May 10, 2007

(54) MULTI-PRONG CONNECTOR, SYSTEM AND METHOD OF USE

(76) Inventor: Duane Kelloway, Spring Hill, FL (US)

Correspondence Address: CHRISTOPHER PARADIES, PH.D., FOWLER WHITE BOGGS BANKER, P.A., 501 E KENNEDY BLVD, STE. 1900, TAMPA, FL, 33602, US

(21) Appl. No.: 11/551,633

(22) Filed: Oct. 20, 2006

Related U.S. Application Data
(60) Provisional application No. 60/729,371, filed on Oct. 21, 2005.

(51) Int. Cl. A61M 5/00; A61B 19/00

(52) U.S. Cl. 604/258; 604/410

(57) ABSTRACT

A surgical irrigator is coupled to a multi-prong connection that is capable of coupling multiple irrigation bags simultaneously. The multi-prong connection comprises a plurality of inlets to a single outlet that is used to supply irrigation fluid to the irrigator. By selecting the length of tubing, diameter of tubing, height of the bags, or a combination of these parameters, a system is designed such that an irrigation bag empties and may be replaced earlier than at least one of the other bags coupled to the irrigator. A system and methods of using such system may be designed to prevent any disruption of irrigation.

### RELATED APPLICATIONS

This application claims the benefit of the filing date of U.S. Provisional Patent Application No. 60/729,371, filed Oct. 21, 2005, the disclosure of which is hereby incorporated herein by reference.

### FIELD OF THE INVENTION

The field relates to irrigators used in medical and surgical procedures, particularly to irrigators used for emergency trauma surgeries.

### BACKGROUND

Currently, in the hospital, irrigators useful for surgical and medical procedures, such as those shown in Henniges et al, in United States Publication No. 2003/0036723, are used to deliver pulses of fluid to a specific site on or in the body of the patient. The fluid comes from an irrigation bag, such as a 3 liter bag, of a sterile saline solution, that is gravity fed or suctioned through a supply tube. The irrigator has an integrated suction line that is capable of removing the fluid, detritus, and bodily fluids from point of irrigation.

Currently, when fluids run out, the irrigator cannot be used before removing the empty bag and replacing the irrigator tube into a new bag, which slows irrigation, requires constant monitoring and provides an opportunity for introduction of infectious organisms. Often, especially in operating room procedures, three or more bags of 3 liters each are used, and require multiple delays during bag replacement. However, the changeover from one bag to the next also delays suction. Thus, it would be advantageous to have a system that does not require interrupting irrigation for replacing empty irrigator bags.

### SUMMARY OF THE INVENTION

A multi-prong connector for use in an irrigation system including an irrigator and a plurality of irrigation bags includes a plurality of inlets, each having an orifice capable of being coupled to one of the plurality of irrigation bags, a junction in fluid communication with each of the plurality of inlets, a control mechanism disposed between at least one of the plurality of inlets and the junction; an outlet in fluid communication with each of the plurality of inlets and tubing coupling each of the plurality of inlets to the junction and the junction to the outlet. In one design, irrigation fluid is capable of flowing through tubing from each of the plurality of irrigation bags, when coupled to one of the plurality of inlets, through the junction and to the irrigator via the outlet, under control of the control mechanism.

In one example, a medical irrigator has a multi-prong connection, such that more than one irrigation bag is capable of being connected simultaneously. Additionally, a system using the multi-prong connection is capable of continuous irrigation and suction without bag replacement or during replacement of one of the bags connected to the multi-prong connection.

One advantage is that irrigation is not interrupted by replacement of an empty bag. Another advantage is that the system is capable of staggering the emptying of multiple bags. Another advantage is that for many procedures, no opportunity is provided for the introduction of pathogens during removal and replacement of a bag. In yet another advantage, the junction may be seamlessly formed with the tubing leading from the inlets to the outlet.

### BRIEF DESCRIPTION OF THE DRAWINGS

The drawings illustrate examples of the present invention.

FIG. 1 illustrates a three-way connector.

FIG. 2 illustrates a tube and a ratchet clamp capable of coupling a connector to an IV bag, such as shown in FIG. 1.

FIG. 3 illustrates another multi-prong connector.

FIG. 4 illustrates a multi-prong connector having three prongs of different diameters joined in an array similar to FIG. 3.

FIG. 5 illustrates yet another example of a connector having three prongs.

FIG. 6 depicts an interconnect element used to couple two components in a system for irrigation.

### DETAILED DESCRIPTION OF PREFERRED EMBODIMENTS OF THE INVENTION

The examples described and the drawings rendered are illustrative and are not to be read as limiting the scope of the invention as it is defined by the appended claims.

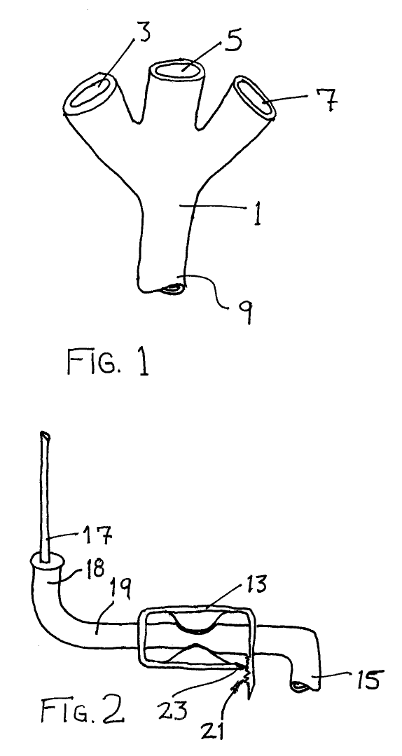

FIG. 1 illustrates a multi-prong connector 1, having three prongs 3, 5, 7. Each prong is capable of being coupled to an irrigation bag, such as by the tube assembly illustrated in FIG. 2. The outlet 9 is capable of being coupled to an irrigator. The connector 1 may be of any geometric shape and size and may include any number of prongs, 3,5,7. For example, 4 prongs may be included. At least 3 prongs are preferred, allowing for three bags to be simultaneously connected to an irrigator. It is believed, without being limiting, that many surgical procedures may be completed using three standard 3-liter irrigation bags. Thus, a multi-prong connector having 3 prongs has the advantage of requiring no disconnection from an emptying bag during many surgical procedures, which limits the introduction of pathogens and avoids interruptions of the irrigating fluid.

A line 19, such as the one shown in FIG. 2 is capable of coupling each of the prongs 3,5,7 to an irrigation bag (not shown) using a sterile, quick connect 17. A tube outlet 15 couples to one of the prongs 3,5, 7. For example, the tube outlet 15 may be coupled by insertion into one of the prongs 3, 5, 7 by an intermediate connector or may be integrally formed with the multi-prong connector 3,5,7 in an one-step or multiple step process. An inlet 18 may have a quick connect coupling 17 which is capable of connecting with an irrigation bag. A clamp 13 that is capable of reducing or stopping flow through the line 19 may be used.

In one example, as shown in FIG. 2, a clamp 13 has teeth 21 and extension 23. The teeth 21 are capable of engaging and disengaging the extensions 23. In the example, the clamp 13 may be ratcheted by pinching the clamp and released by disengaging the portion having teeth 21 from the extension 23. A junction, such as illustrated in FIGS. 1, 3, 4 and 5 may be seamlessly joined with the tubing of FIG. 2, such that a seamless system is capable of providing irrigation fluid flow from at least three irrigation bags to an irrigator during a surgical procedure. In an alternative example, the junction has connectors for connecting to the tubing leading to the inlets and outlet.

The flow rate through a tube depends on the pressure drop, on the radius of the tube to the fourth power and inversely on the tube length. The hydrodynamic resistance to flow may be thought of as a resistance to current flow, which is proportional to length divided by radius to the fourth power.

In one example of the present invention, a multiple coupler with three prongs is connected to three irrigation bags with different tube lengths. A longer tube has greater resistance to flow. Thus, the longest tube length will have the lowest flow rate. In one example, each of the bags is mounted on a hook at the same height; therefore, there is little contribution to the flow rate by any difference in the hydrostatic pressure. In another example, the bags are mounted at different heights relative to one another. For example, the bag connected by the shortest tube may be connected on the highest hook; therefore, the flow rate is influenced by both a lower resistance to flow and a greater pressure drop applied across the tube.

If the hydrodynamic resistance is sufficiently large, then the contribution to the pressure drop with small changes in the hydrostatic head (i.e., ρg Δh, where ρ is fluid density, g is the gravitational constant and Δh is the difference in height) may be neglected. In this case, only changes in the length of the tube and the tube diameter will be significant in determining the flow rate.

In one example of the present invention, the tube diameter of each tube is selected to control the flow rate that the flow rate may be greater in the first tube, less in a second tube, and least in a third tube, if the first tube diameter is greater than the second tube diameter, which is greater than the third tube diameter.

Thus, if three bags containing a fluid of equal viscosity and volume are coupled to each of three tubes and the height of the fluids in the bags may be neglected, then the bag connected to the first tube will empty its contents prior to contents of the bag connected to the second tube if the tube length and/or diameter is approximately selected for each tube. The contents of the bag connected to the second tube may be selected to empty prior to the contents of the bag connected to the third tube. In one example, the length of a line 19 is chosen such that at least one bag is always emptied prior to the emptying of one of the other bags. Then, a line 19 connecting the empty bag may be closed using the clamp 13, and the empty bag may be replaced without interrupting the supply of irrigation fluid during irrigation.

In another example, the tube diameter of at least one of the lines 19 is selected to be larger than the tube diameter of another of the lines 19. Thus, irrigation flows through the larger diameter line 19 at a higher flow rate, which empties a bag of equal volume more quickly.

In yet another example, at least one of lines 19 is selected to have both a shorter length and a larger diameter than the other lines 19. Thus, the flow rate is greater for fluid flow such that a volume of fluid empties through the line 19 more quickly than the other lines 19.

If the resistance to fluid flow through a line 19 is small compared to the force induced by a difference in hydrostatic head (i.e., ρg Δh, where ρ is fluid density, g is the gravitational constant and Δh is the difference in height), then the hydrostatic head may not be neglected.

A difference in height, Δh, may be imposed by positioning at least one of the bags at a height greater than the height of the other bags. In one example, a system couples a line having the greatest flow rate (least flow resistance) to a bag having the largest height difference. Therefore, the flow rate is greater through this tube than through other tubes connected to a multi-prong connector 1 such that a bag of equal volume to the other bags that is coupled to this line will always empty first. Selection of a tube diameter D, line length, L, and a hydrostatic head Δh may be used either combined or separately in a system such that at least one bag empties prior to one of the other bags, assuming each of the bags contains the same volume of the fluid.

In one example, the line 19 length and the line 19 diameter of each of the lines 19 used in a system with a multi-prong connector 1 are selected such that each of the bags connected by the multi-prong connector 1 empties in a sequential order.

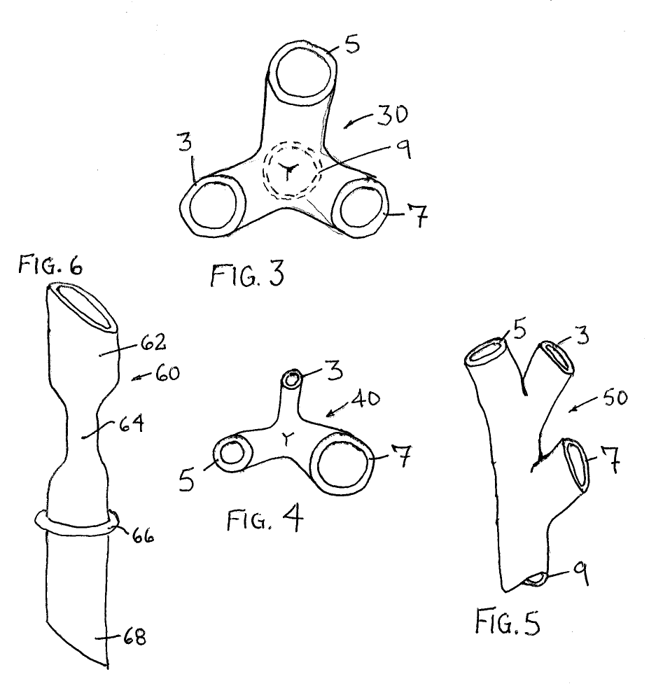

In FIG. 3, the multi-prong connector 30 has three prongs 3,5,7 that are connected in a three-dimensional array merging three inlet lines into one outlet line 9 (hidden dashed line) opposite of the three inlet lines. The inlets of FIG. 3 are tetrahedrally arranged. In contrast, the inlet prongs 3,5,7 and outlet prong 9 of the connector I of FIG. 1 are illustrated as lying in a common plane.

In FIG. 4, the largest diameter prong 7 has a lesser resistance to flow than either of the smaller diameter prongs 3,5. Thus, all else being equal, a bag connected to the largest diameter prong 7 will empty first, allowing this bag to be replaced prior to the emptying of the other bags that may be connected to other prongs, 3,5.

In yet another example of a connector 50 having multiple prongs 3,5,7, as shown in FIG. 5, two of the prongs 3,5 are connected in a Y connection. Then, the outlet of the Y connection is coupled to a third prong 7. In one example, the Y connection is coupled to a third prong 7. In one example, the connector 50 is integrally formed and seamless. In another example, the connector 50 is formed by joining two Y connectors, using an intermediate interconnection from the exit of one Y connector to the inlet of a second Y connector. The length and/or diameter of the intermediate connection may be selected to increase the flow resistance for fluid through inlets 3,5 of a first connector and the inlet 7 of a second connector, for example. Likewise, a 5-prong connectors (not shown) may be formed by joining two 3-prong connectors 1, 30, 40, and a 4-prong connector (not shown) may be formed by coupling a 3-prong connector and a 2-prong connector.

In one example, a system uses a multi-prong connector 50 having an intermediate connection of a length and diameter selected such that a fixed volume of fluid necessarily empties through at least one of the inlets 7 before the same fixed fluid empties through another of the inlets 3,5. Thus, an irrigation bag may be replaced without interrupting irrigation.

In yet another example as illustrated in FIG. 6, an interconnect 60 is used to couple two components in a system for irrigation. For example, FIG. 6 shows an interconnect 60 having a first end 62 with a constriction 64, a flange 66 and a second end 68. Two tubes 19, two connectors 1,30, 40,50, or a tube 19 and a connector 1, 30, 40, 50 may be connected by the interconnect 60. The constriction 64 may be used to increase the resistance to fluid flow to one or more of the bags coupled to the irrigator. Thus, a system may use the interconnect 60 to prevent interruption of irrigation.

Alternative combinations and variations of the examples provided will become apparent based on this disclosure. It is not possible to provide specific examples for all of the many possible combinations and variations of the embodiments described, but such combinations and variations may be claims that eventually issue.

What is claimed is:

1. A multi-prong connector for use in an irrigation system including an irrigator and a plurality of irrigation bags, comprising:
a plurality of inlets, each having an orifice capable of being coupled to one of the plurality of irrigation bags;
a junction in fluid communication with each of the plurality of inlets;
a control mechanism disposed between at least one of the plurality of inlets and the junction;
an outlet in fluid communication with each of the plurality of inlets; and a tubing coupling each of the plurality of inlets to the junction and the junction to the outlet, such that irrigation fluid is capable of flowing through the tubing from each of the plurality of irrigation bags, when coupled to one of the plurality of inlets, through the junction and to the irrigator via the outlet, under control of the control mechanism.

2. The multi-prong connector of claim 1, wherein the control mechanism is selected from the group of control mechanisms consisting of decreasing the diameter of at least a portion of the tubing between the orifice of at least one of the plurality of inlets and the junction compared to the diameter of tubing between the orifice of another of the plurality of inlets and the junction; a valve; a clamp; and a combination thereof.

3. The multi-prong connector of claim 2, wherein the control mechanism is a clamp.

4. The multi-prong connector of claim 3, wherein the claim is a ratchet clamp.

5. The multi-prong connector of claim 1, wherein the plurality of inlets is at least three inlets.

6. The multi-prong connector of claim 5, wherein the plurality of inlets meet at the junction and are arranged tetrahedrally.

7. The multi-prong connector of claim 1, wherein the plurality of inlets meet at the junction, and the tubing from each of the inlets lies in a common plane.

8. The multi-prong connector of claim 1, wherein the tubing and the junction are seamlessly formed.

9. The multi-prong connector of claim 1, wherein the tubing comprises a first tube and a second tube, the first tube coupling a first of the plurality of the inlets to the junction and having a first length, the second tube coupling a second of the plurality of the inlets to the junction and having a second length, wherein the first length is greater than the second length.

10. The multi-prong connector of claim 9, wherein the first tube has a reduced diameter along at least a portion of the first tube, and the second tube has a second diameter, and the second diameter is greater than the reduced diameter of at least a portion of the first tube.

11. A method of irrigating for use in surgical procedures, using a multi-prong connector, a plurality of irrigation bags, and an irrigator, the method comprising:
connecting the plurality of irrigation bags containing an irrigation fluid to a plurality of tubes using the multi-prong connector, wherein the multi-prong connector comprises a plurality of inlets, each of the plurality of inlets having an orifice coupling one of the plurality of irrigation bags;
a junction coupled by tubing to each of the plurality of inlets and in fluid communication with each of the plurality of inlets;
a control mechanism disposed between at least one of the plurality of inlets and the junction; and an outlet coupled by tubing to the junction and in fluid communication with each of the plurality of inlets, allowing irrigation fluid flow through the tubing from each of the plurality of irrigation bags, when coupled to one of the plurality of inlets, through the junction and to the irrigator via the outlet; and
controlling the irrigation fluid flow through the tubing of the plurality of inlets using the control mechanism of the multi-prong connector, such that one of the plurality of irrigation bags empties before another of the plurality of irrigation bags empties, during use.

12. The method of claim 11, wherein the control mechanism is a ratchet clamp and the step of controlling comprises clamping the ratchet clamp on the tubing between one of the plurality of inlets and the junction, such that the flow of fluid is impeded from one of the irrigation bags; and releasing the ratchet clamp before another of the irrigation bags is emptied, such that the flow of irrigation fluid to the irrigator is not interrupted.
